(19) United States
(12) Patent Application Publication
(10) Pub. No.: US 2022/0257372 A1
(43) Pub. Date: Aug. 18, 2022

(54) IMPLANT, DELIVERY DEVICE AND METHOD OF DELIVERING AN IMPLANT

(71) Applicant: P+F CARDIOVASCULAR GMBH, Wien (AT)

(72) Inventors: Siegfried Einhellig, Munich (DE); Guilherme Agreli, Sao Paolo (BR); Katharina Kiss, Wien (AT)

(21) Appl. No.: 17/625,565

(22) PCT Filed: Jul. 10, 2020
(86) PCT No.: PCT/EP2020/069560

§ 371 Date: Jan. 7, 2022

(30) Foreign Application Priority Data
Jul. 10, 2019 (EP) 19185491.8

(51) Int. Cl. A61F 2/24; A61F 2/95; A61F 2/90

(52) U.S. Cl. CPC A61F 2/2418; A61F 2/2427; A61F 2/9517; A61F 2250/0069; A61F 2220/0025; A61F 2250/0039; A61F 2/90

(57) ABSTRACT

The present invention relates to an implant for placement in a cardiac vessel, the implant being able to be expanded from an un-deployed state to an expanded state in which the implant has a larger outer diameter than in the storage state, the implant having a main body with first and second ends and a central portion arranged between the first and second ends, the central portion of the implant having a larger outer diameter than the first and second ends of the implant when the implant is in the expanded state. The invention further relates to a delivery device for an implant and to a method of delivering an implant.

The present invention relates to an implant for placement in a cardiac vessel, especially an implant for aortic root reconstruction and aortic valve replacement, the implant being able to be expanded from an un-deployed state to an expanded state in which the implant has a larger outer diameter than in the storage state, the implant having a main body with first and second ends and a central portion arranged between the first and second ends, the central portion of the implant having a larger outer diameter than the first and second ends of the implant when the implant is in the expanded state. The invention further relates to a delivery device for an implant and to a method of delivering an implant.

Patients suffering from acute type A dissection, ascending aorta aneurysm, or other conditions that require urgent intervention, such as, ascending aorta replacement are frequently subjected to the so-called Bentall-de Bono procedure during which a part of the ascending aorta, the aortic valve, and the aortic root is removed while a patient is placed on bypass.

Following this a replacement valve is fitted and a graft is sewn in place at the aortic valve together with the replacement valve. Thereafter a portion of the ascending aorta is attached to the graft and the coronary arteries are likewise sewn into the graft.

Such surgical procedures generally take several hours to perform and the re-implantation of the coronary arteries is prone to complications. Moreover, the time to treatment is a crucial factor in reducing the mortality rate of acute aortic syndromes, i.e. a reduction in the time to treatment can lead to a reduced mortality rate. It has also been found that the replacement of the aortic root should be avoided as far as possible, since this can lead to further complications.

For this reason it is an object of the present invention to make available a device in ascending aorta reconstruction with aortic valve replacement, as an endovascular/percutaneous/minimally invasive alternative to the Bentall-de Bono procedure by means of which the complications which may arise during the invasive Bentall-de Bono procedure can be minimized. It is a further object of the present invention to reduce the time to treatment of acute aortic syndromes.

This object is satisfied by an implant having the features of claim 1.

Such an implant for placement in a cardiac vessel is able to be expand from an un-deployed state to an expanded state in which the implant has a larger outer diameter than in the storage state, with the implant having a main body with first and second ends and a central portion arranged between the first and second ends, the central portion of the implant having a larger outer diameter than the first and second ends of the implant when the implant is in the expanded state, the implant further comprising:
a replacement valve for placement at the aortic valve arranged at the first end of the implant,
a self-expandable aortic stent frame for placement at the ascending aorta arranged at a second end of the implant; and
two or more internal branches for connection to further vessels, the internal branches extending from the implant.

The implant is thus a combination of a transcatheter aortic valve, an ascending aorta stent graft, and branches configured to be connected to covered coronary stents. Thereby the number of complications which may arise during the attachment of the coronary arteries and the aortic root to the stent graft on performing the Bentall-de Bono procedure can be reduced.

Moreover, a patient does not have to be placed on bypass during the procedure which further reduces the possibility of bleeding, arrhythmias, infections of the chest wound, memory loss, kidney problems, stoke or even a heart attack.

The implant is designed to be implanted using the arterial femoral access which can be conducted minimally invasive. In this way the recovery time of a patient can be significantly reduced leading to reduced cost of hospitalization.

The implant may be reliably used to treat patients suffering from acute type A dissection, ascending aorta aneurysm, or other conditions that require urgent intervention.

By way of such a device the time to treatment can be significantly reduced leading to reduced mortality rates for acute aortic syndromes, provided the patient is within the vicinity of a medical facility. This is because the valve replacement device in the form of the implant can save time to treatment when this is transported to the heart via the arterial femoral access. This is particularly the case if the valve is preloaded at a delivery device and the equipment is ready to use from a shelf.

The main body of the implant may be completely covered with a polymeric or biological material, optionally wherein the internal branches and in particular the complete implant is covered with material. In this way damage to the tissue at which the implant is placed can be avoided, reducing the number of complications that could be present without such a layer of material.

Respective first exits of the internal branches may have an opening that may into the central portion of the implant and respective second exits of the internal branches may have an opening that may open into an area surrounding the implant. In this way a blood flow stemming from the coronary arteries can be introduced into the implant without having to cut the arteries from the aortic root on treating acute type A dissection, ascending aorta aneurysm, or other conditions that require urgent intervention.

The valve may be a biological dry tissue valve. Such a valve allows the preloading of the implant within a delivery system. Such procedures save time during implantation and increase safety, avoid making mistakes or the provocation of failures during the loading of the implant at the delivery system.

The replacement valve may comprise between two and five leaflets. Such valves have been found to be beneficial when placed at the aortic valve.

A respective anti-leakage skirt may surround the first and second ends. Such anti-leakage skirts, on the one hand facilitate the function of the respective part of the implant and also allow an improved attachment of the implant at the respective vessel to which it is intended to be attached.

The first end may have an outer diameter that is smaller than an outer diameter of the second end in the expanded state. Such diameters have been found to be required for accurate placement at the aortic valve and the aortic root.

A diameter of the implant may decrease between the first end and the central portion and a diameter of the implant may also decrease between the second end and the central portion in the expanded state. By forming the implant in this way an attachment of the implant at the aortic root can be improved.

An outer diameter of the main body may reduce in size between the first end up to a first point of transition and then increases in size toward the part of the central portion having the largest outer diameter, with the outer diameter of the main body decreasing in size from the part of the central portion having the largest outer diameter up to a second point of transition, and with the outer diameter of the main body increasing in size between the second point in transition and the second end. Such a shape is believed to be particularly suitable for a minimally invasive treatment of acute type A dissection, ascending aorta aneurysm, or other conditions that require urgent intervention.

The main body may comprise two stent frames with the first stent frame having the replacement valve attached therein and the second stent frame comprising the self-expandable aortic stent frame, the first stent frame extending from the first end to beyond the part of the central portion having the largest outer diameter.

The expansion of the implant may be made possible by the respective first and second stent frames used. The respective first and second stent frames may each include a plurality of arms that are interconnected in such a way that following the expansion of the implant into the expanded state they adapt to the anatomical need of the location of the implant. In this way the design of such a stent frame is adapted to accommodate the anatomical needs and implantation locations.

The first and second stent frames may e.g. comprise e.g. a balloon or self-expandable stent frame. Moreover, between 6 and 50 arms may be provided to form the respective stent frames.

Similarly the internal branches may comprise further stent frames which can be deployed in the coronary arteries adjoining the aortic valve. The stent frames of the internal branches may likewise be formed with a plurality of arms and from the same or similar materials as the first and second stent frames.

In this connection it should be noted that each of the first and second stent frames may be designed from a collapsible and flexible metal. The material can in this respect be superelastic, a shape memory alloy, self-expandable (e.g. Nitinol), balloon expandable (e.g. Cobalt chromium, Stainless Steel, etc.) or polymeric or artificial. In order to manufacture the first and second stent frames, these can be laser cut form a piece of tube, the first and second stent frames can also be braided.

Additionally or alternatively the different sections of the first and second stent frames can be made of different materials or continuous material, with the different materials then being selected in dependence on the desired purpose. For example, the first stent frame for placement at the ascending aorta may be designed more flexible than the second stent frame as this is designed to hold and support the replacement valve at the aortic valve.

Additional anchoring of the implant to an artery can be accomplished by means of sutures, barbs or hooks that can be further provided at the respective first and second stent frame. Moreover, a radial force present in each section of the first and second stent frames can be adjusted via the materials selected for each section of the respective stent frame, the specific (outer) dimensions selected for each section of the respective stent frame or the processing of the specific sections may be dimensioned for placement at the location of the aortic valve and the ascending aorta.

The first stent frame like the second stent frame may comprises one or more eyelets for fixing the stent frame at the ascending aorta and the aortic valve respectively.

The first stent frame may extend from the first end to a height of the implant corresponding to 55% to 75%, in particular to 60% to 70%, especially to 62% to 68%, of a length of the implant between an opening at the first end of the implant and an opening at the second end of the implant.

The second stent frame may extend from an opening at the second end to the second transition. A length of the second stent frame may correspond to a length of 15 to 35%, in particular 20 to 30% and especially of 22 to 28% of a length of the implant between the opening at the second end and the opening at the first end.

A gap present between the first stent frame and the second stent frame may have a width that corresponds to 2 to 30%, in particular 5 to 20%, especially 6 to 15%, of a length of the implant between the opening at the second end and the opening at the first end.

Such lengths, sizes and dimensions of the respective sections are believed to be particularly suitable for a minimally invasive treatment of acute type A dissection, ascending aorta aneurysm, or other conditions that require urgent intervention.

The first exits from the internal branches may open into the central portion of the implant between the first point of transition and the part of the central portion having the largest outer diameter, in particular:

wherein openings of the first exits of the internal branches may have a diameter which corresponds to 30% to 70% of a length of the implant between the part of the central portion having the largest outer diameter and the first point of transition; and/or
wherein openings of the first exit from the internal branches may be arranged between 20% to 80% of a length of the implant between the part of the central portion having the largest outer diameter and the first point of transition.

Arranging the exits from the internal branches in this way is believed to be beneficial to conducting the minimally invasive surgery envisaged for this implant.

The first end may have an at least substantially cylindrical outer shape between an opening of the implant and the part of the main body reducing in size between the first end and the first point of transition, optionally wherein the first end may have an at least substantially cylindrical outer shape at an opening of the replacement valve, in particular wherein the replacement valve may be arranged within the at least substantially cylindrical outer shape. Such shapes are known to be beneficial as shapes of valves used to replace the aortic valve.

The two or more internal branches may be arranged at the main body between the central portion and the replacement valve arranged at the first end of the implant. in this way the attachment of the branches to covered coronary stents installed in the coronary arteries can be simplified and facilitated.

The two or more internal branches may be arranged at the main body between the first point of transition and the part of the central portion having the largest outer diameter. This further facilitates the attachment of the internal branches to the covered coronary stents installed in the coronary arteries.

According to a further aspect the above object is also satisfied by delivery device for an implant, the delivery device comprising;

a flush port;

a main body part for holding, inflating and/or releasing the implant; one or more guidewires for holding, inflating and/or releasing one or more internal branches of the implant; and

an actuation mechanism for moving the implant to a delivery site.

The delivery device may have an apparatus to pre-catheterize the internal branches that are used to connect the coronary arteries to the implant. Such an apparatus saves time during the procedure, avoiding manipulation of guidewires and catheters inside the implanted implant.

The delivery device may be preloaded with an implant so that this can be stored ready to use on a shelf in a medical facility to significantly reduce the treatment time of acute aortic syndromes leading to reduced mortality rates of acute aortic syndromes.

The implant and/or the delivery device may have one or more radiopaque markings to increase visibility under x-rays and to increase precision and accuracy during deployment.

The one or more guidewires can be retracted from the main body on pre-catheterization of the internal branches at the delivery site.

The actuation mechanism may have a torque control and may be able to rotate the implant about an axis of the main body. In this way the implant can be positioned in relation to the extremities in an as good as possible manner at the aortic valve, to deploy the valve and stent at the desired deliver site.

The delivery device may be improved with steerable control, to increase precision and accuracy at deployment.

The delivery device may have a knob or the like at the actuation mechanism, with the knob in particular being able to be rotated about an axis of rotation of the actuation mechanism. By turning the knob the lumen is able to deflect and allows a better positioning of the tip and the main body of the delivery device and less stress over the system during deployment. This leads to an improved accuracy of deployment of the delivery device and hence of an implant that is delivered to a delivery site using the delivery device.

The one or more guidewires can be retracted from the main body via two valved introducers installed at the delivery device. One or more diagnostic catheters can be replaced via the valved introducers, so that the implant can be positioned as accurately as possible at the implant target site.

The valved introducers are connected to the actuation mechanism and preferably guided through the lumen to the tip. This ensures a good connection and guidance of both diagnostic catheters and guidewires.

The flush port is arranged between the valved introducers and the knob of the actuation mechanism. This ensures a design of the delivery device that is as simple as possible in its use.

According to a further aspect the above object is also satisfied by a method of delivering an implant using a delivery device, the method comprising the steps of:
moving the implant to a delivery site by means of the delivery device;
pre-catheterizing the internal branches of the implant at the delivery site;
positioning the main body of the implant at the delivery site; and
deploying the implant at the delivery site.

Such a minimally invasive procedure is believed to significantly reduce the complications known from the Bentall-de Bono procedure.

The invention will be described in detail by means of embodiments and with reference to the drawings. These show preferred deployment locations and embodiments. The features described may be configured in various combinations, which are encompassed in this document. The drawings show:

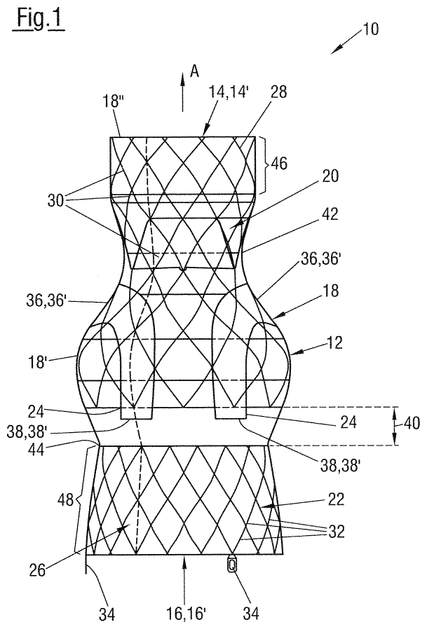

FIG. 1 a side view of an implant;

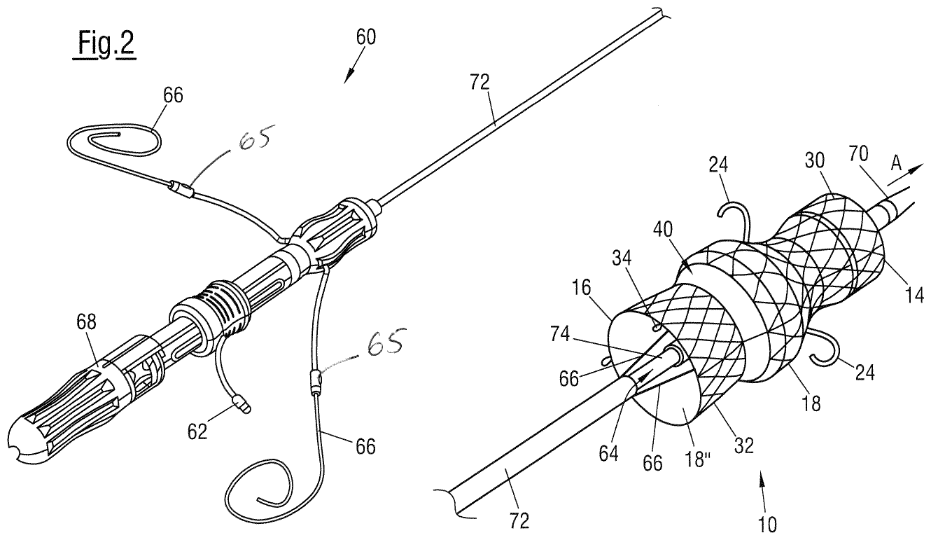

FIG. 2 a perspective view of a delivery device having an implant arranged thereat; and

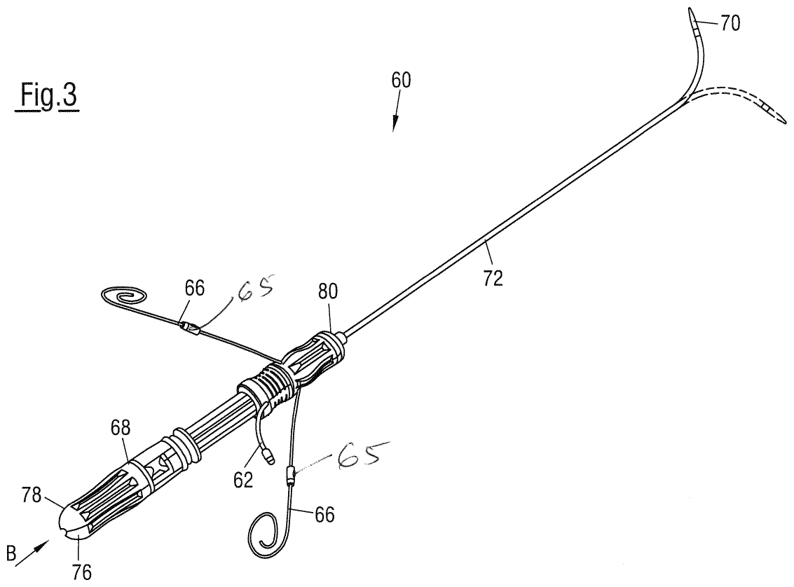

FIG. 3 a further perspective view of the delivery device of FIG. 2 showing positions a tip thereof can occupy when an actuation mechanism of the delivery device is activated.

In the following the same reference numerals will be used for parts having the same or equivalent function. Any statements made having regard to the direction of a component are made relative to the position shown in the drawing and can naturally vary in the actual position of application.

FIG. 1 shows an implant 10 for placement in a cardiac vessel, the implant 10 being able to be expand from an un-deployed state to an expanded state in which the implant 10 has a larger outer diameter than in the storage state. The implant 10 has a main body 12 with first and second ends 14, 16. A central portion 18 is arranged between the first and second ends 14, 16. The central portion 18 of the implant 10 has a larger outer diameter than the first and second ends 14, 16 of the implant 10 respectively when the implant 10 is in the expanded state.

The implant 10 further comprises a replacement valve 20 for placement at the aortic valve arranged within the first end 14 of the implant 10. A passage 18″ extends from the first end 14 to the second end 16, with the replacement valve 20 being arranged within the passage 18″.

A self-expandable aortic stent frame 22 for placement at the ascending aorta is arranged within the second end 16 of the implant 10. Furthermore, two internal branches 24 extend from the main body 12. The internal branches 24 are configured to be attached to either the coronary arteries, or if provided a respective stent arranged within the coronary arteries (both not shown).

The implant is completely covered with a material 26. The material 26 covers two stent frames 22, 28 with the first stent frame 28 having the replacement valve 20 attached therein and the second stent frame 22 comprising the self-expandable aortic stent frame.

The first stent frame 28 extends from an opening 14′ of the first end 14 to beyond a part 18′ of the central portion 18 having the largest outer diameter. A gap 40 is present between the first stent frame 28 and the second stent frame 22, with the material 26 bridging the gap 40 between the first and second stent frames 28, 22.

Each of the first and second stent frames 22, 28 comprises a plurality of respective arms 30, 32 which are indicated in a shaded manner in FIG. 1. The arms are interconnected to form the respective expandable stent frame 22, 28. The second stent frame 22 extends from an opening 16′ at the second end 16 to the second transition 44.

Eyelets 34 are visible at the second end. The eyelets 34 are provided at the second stent frame 22 in order to facilitate the attachment of the implant 10 to the aortic root.

The valve 20 may be a dry tissue valve and may, for example, comprise between two and five leaflets. In order to prevent leaks from arising between the valve 20 and the part of the aorta at which it is placed, the implant 10 comprises an anti-leakage skirt 46 that is arranged to cover that part of the first end 14 in which the valve 20 is arranged.

Similarly an anti-leakage skirt 48 may be arranged to surround that part of the second end 16 which is configured to contact the aortic root.

The anti-leakage skirt 48, like the anti-leakage skirt 46 may be formed from one and the same material 26, the material 26 may then extend on an outer surface of the respective first and second stent frames 28, 22 between the first end 14 and the second end 16.

The material 26 may also cover the internal branches 24, so that the complete implant 10 is covered with one piece of material 26 that then preferably does not comprise any holes, leaks or the like.

The first end 14 has an outer diameter that is smaller than an outer diameter of the second end 16 in the expanded state, with an outer diameter of the main body 12 reducing in size between the first end 14 up to a first point of transition 42 and then increases in size toward the part 18′ of the central portion 18 having the largest outer diameter, with the outer diameter of the main body 12 decreasing in size from the part 18′ of the central portion 18 having the largest outer diameter up to a second point of transition 44. The outer diameter of the main body 12 then increases in size between the second point 44 in transition and the second end 16.

The first end 14 of the implant 10 can be defined as that part of the implant that extends between the opening 14′ and the first point of transition 42.

The central portion of the implant 10 can be defined as that part of the implant 10 that extends between the first point of transition 42 and the second point of transition 44.

The second end 16 of the implant 10 can be defined as that part of the implant 10 that extends between the second point of transition 44 and the opening 14′.

The internal branches 24 each have a first exit 36 with an opening 36′ and a second exit 38 with an opening 38′. The openings 36′ each open into the central portion 18 of the implant 10. The openings 38′ present at the second exits 38 each open into an area surrounding the implant 10.

The first exits from the internal branches 24 open into the central portion 18 of the implant 10 between the first point of transition 42 and the part of the central portion 18 having the largest outer diameter.

The first end 14 has an at least substantially cylindrical outer shape between the opening 14′ of the implant 10 and the part of the main body 12 reducing in size between the first end 14 and the first point of transition 42.

FIG. 2 shows a delivery device 60 for the implant 10 also known as a tri-axial catheter. The delivery device 60 comprises a flush port 62 for flushing internal cavities of the delivery device (not shown), a main body 64 for holding, inflating and/or releasing the implant 10, two valved introducers 65 to allow access to the branches 24 of the implant 10. Initially two diagnostic catheters (not shown) are introduced via the two valved introducers 65 together with the preloaded implant 10 for delivering the implant 10 to the target delivery site, i.e. the patients' heart.

Once the diagnostic catheters wires have been used they are each removed via the respective one of the two valved introducers 65 and replaced with guidewires 66 for holding, inflating and/or releasing one or more internal branches 24 of the implant 10, and an actuation mechanism 68 for moving the implant 10 to a delivery site.

The main body 64 is arranged at an inner shaft 74 at an end of the lumen 72 that extends between the actuation mechanism 68 and a tip 70 of the tri-axial catheter 60. On delivery of the implant 10 at the delivery site, the internal branches are pre-catheterized, e.g. using a balloon catheter and are thereby connected to the coronary arteries and/or stent frames present in the coronary arteries. Following the attachment of the internal branches 24 at the coronary arteries the guidewires 66 can be retracted from the main body 64.

The implant 10 can be guided to the delivery site by means of the actuation mechanism 68 that has a torque control and can rotate the implant 10 about an axis A of the main body 12. The axis A extends between the openings 14′, 16′ of the main body 12 of the implant 10.

FIG. 3 shows a perspective view of the delivery device 60 of FIG. 2. The actuation mechanism 68 comprises a knob 76 at a distal end 78 of the actuation mechanism 68. On turning the knob 68 about an axis B of the actuation mechanism 68 which extends between the distal end 78 and a proximal end 80 of the actuation mechanism 68, the lumen 72 is able to deflect. This enables the tip 70 of the lumen 72 to occupy various positions, of which two are indicated in FIG. 3. Such a deflection of the lumen 72 reduces the overall stress in the system which leads to a better positioning of the implant 10 at a delivery site and an overall improved accuracy of deployment of the delivery device 60 and hence of an implant 10 that is delivered to a delivery site using the delivery device 60.

On delivering the implant 10 to the delivery site, i.e. the heart, by means of the delivery device 60, the arterial femoral access is preferably used. The lumen 72 is then moved through the arteries to the heart.

The position of the implant 10, the diagnostic catheters and/or of the delivery device 60 may be tracked using e.g. x-rays in order to monitor radiopaque markings present at the implant 10 and/or the delivery device 60. Thereby the precision and accuracy during deployment of the implant 10 within the heart can be further increased, so that the replacement valve 20 can be positioned accurately at the aortic valve. Once the implant 10 is positioned correctly, the diagnostic catheters are each removed via the respective valved introducer 65 and replaced with the guidewires 66 that are subsequently used to catheterize the coronary arteries and to install the coronary stents.

### LIST OF REFERENCE NUMERALS

10 implant
12 main body
14, 14′ first end, opening at 14
16, 16′ second end, opening at 16
18, 18′, 18″ central portion, part of 18 laving largest outer diameter of 10, passage
20 valve
22 second stent frame
24 branches
26 material
28 first stent frame
30 arms
32 arms
34 eyelets
36, 36′ first exit, opening of 36
38, 38′ second exit, opening of 38
40 gap
42 first point of transition
44 second point of transition
46 anti-leakage skirt
48 anti-leakage skirt
60 delivery device
62 flush port
64 main body part of 60
66 guidewire
68 actuation mechanism
70 tip
72 lumen
74 inner shaft
76 knob
78 distal end
A axis of 10
B axis of 68

1. An implant for placement in a cardiac vessel, the implant being able to be expanded from an un-deployed state to an expanded state in which the implant has a larger outer diameter than in the storage state, the implant having a main body with first and second ends and a central portion arranged between the first and second ends, the central portion of the implant having a larger outer diameter than the first and second ends of the implant when the implant is in the expanded state, the implant further comprising:
a replacement valve arranged at the first end of the implant for placement at the aortic valve,
a self-expandable aortic stent frame arranged at the second end of the implant for placement at the ascending aorta; and
two or more internal branches for connection to further vessels, the internal branches extending from the main body.

2. The implant according to claim 1, wherein the main body is completely covered with a material.

3. The implant according to claim 2, wherein at least one of the internal branches and the complete implant is covered with material.

4. The implant according to claim 1, wherein respective first exits of the internal branches have an opening that opens into the central portion of the implant and respective second exits of the internal branches have an opening that opens into an area surrounding the implant.

5. The implant according to claim 1, wherein the valve is a dry tissue valve.

6. The implant according to claim 1, wherein a respective anti-leakage skirt surrounds the first and second ends.

7. The implant according to claim 1, wherein the replacement valve comprises between two and five leaflets.

8. The implant according to claim 1, wherein the first end has an outer diameter that is smaller than an outer diameter of the second end in the expanded state.

9. The implant according to claim 1, wherein a diameter of the implant decreases between the first end and the central portion and between the second end and the central portion in the expanded state.

10. The implant according to claim 1, wherein an outer diameter of the main body reduces in size between the first end up to a first point of transition and then increases in size toward the part of the central portion having the largest outer diameter, with the outer diameter of the main body decreasing in size from the part of the central portion having the largest outer diameter up to a second point of transition, and with the outer diameter of the main body increasing in size between the second point in transition and the second end.

11. The implant according to claim 1, wherein the main body comprises two stent frames with the first stent frame having the replacement valve attached therein and the second stent frame comprising the self-expandable aortic stent frame, the first stent frame extending from the first end to beyond the part of the central portion having the largest outer diameter.

12. The implant according to claim 11, wherein the first stent frame extends from the first end to a height of the implant corresponding to 55% to 75%, in particular to 60% to 70%, especially to 62% to 68%, of a length of the implant between an opening at the first end of the implant and an opening at the second end of the implant.

13. The implant according to claim 11, wherein the second stent frame extends from an opening at the second end to the second transition.

14. The implant according to claim 11, wherein a length of the second stent frame corresponds to a length of 15 to 35% of a length of the implant between the opening at the second end and the opening at the first end.

15. The implant according to claim 11, wherein a gap present between the first stent frame and the second stent frame has a width that corresponds to 2 to 30% of a length of the implant between the opening at the second end and the opening at the first end.

16. The implant according to claim 15, wherein the gap between the first and second stent frames is bridged with material.

17. The implant according to claim 11, wherein respective first exits of the internal branches have an opening that opens into the central portion of the implant and respective second exits of the internal branches have an opening that opens into an area surrounding the implant and wherein the first exits from the internal branches open into the central portion of the implant between the first point of transition and the part of the central portion having the largest outer diameter.

18. The implant according to claim 17, wherein openings of the first exits of the internal branches have a diameter which corresponds to 30% to 70% of a length of the implant between the part of the central portion having the largest outer diameter and the first point of transition.

19. The implant according to claim 17, wherein openings of the first exit from the internal branches are arranged between 20% to 80% of a length of the implant between the part of the central portion having the largest outer diameter and the first point of transition.

20. The implant according to claim 10, wherein the first end has an at least substantially cylindrical outer shape between an opening of the implant and the part of the main body reducing in size between the first end and the first point of transition.

21. The implant according to claim 20, wherein the first end has an at least substantially cylindrical outer shape at an opening of the replacement valve.

22. The implant according to claim 1, wherein the two or more internal branches are arranged at the main body between the central portion and the replacement valve arranged at the first end of the implant.

23. The implant according to claim 22, wherein an outer diameter of the main body reduces in size between the first end up to a first point of transition and then increases in size toward the part of the central portion having the largest outer diameter, with the outer diameter of the main body decreasing in size from the part of the central portion having the largest outer diameter up to a second point of transition, and with the outer diameter of the main body increasing in size between the second point in transition and the second end; and
wherein the two or more internal branches are arranged at the main body between the first point of transition and the part of the central portion having the largest outer diameter.

24. A delivery device for an implant, the implant being able to be expanded from an un-deployed state to an expanded state in which the implant has a larger outer diameter than in the storage state, the implant having a main body with first and second ends and a central portion arranged between the first and second ends, the central portion of the implant having a larger outer diameter than the first and second ends of the implant when the implant is in the expanded state, the implant further comprising:
a replacement valve arranged at the first end of the implant for placement at the aortic valve,
a self-expandable aortic stent frame arranged at the second end of the implant for placement at the ascending aorta; and
two or more internal branches for connection to further vessels, the internal branches extending from the main body;
the delivery device comprising;
a flush port;
a main body part for holding, inflating and/or releasing the implant; one or more guidewires for holding, inflating and/or releasing one or more internal branches of the implant; and
an actuation mechanism for moving the implant to a delivery site.

25. The delivery device according to claim 24, wherein the one or more guidewires can be retracted from the main body on pre-catheterization of the internal branches at the delivery site.

26. The delivery device according to claim 25, wherein the actuation mechanism has at least one of a torque control and can rotate the implant about an axis of the main body and a knob that can be rotated about an axis of rotation of the actuation mechanism.

27. The delivery device according to claim 26, wherein the one or more guidewires can be retracted from the main body via two valved introducers installed at the delivery device.

28. The delivery device according to claim 27, wherein the valved introducers are connected to the actuation mechanism.

29. The delivery device according to claim 28, wherein the flush port is arranged between the valved introducers and the knob of the actuation mechanism.

30. A method of delivering an implant for placement in a cardiac vessel, the implant being able to be expanded from an un-deployed state to an expanded state in which the implant has a larger outer diameter than in the storage state, the implant having a main body with first and second ends and a central portion arranged between the first and second ends, the central portion of the implant having a larger outer diameter than the first and second ends of the implant when the implant is in the expanded state, the implant further comprising:
a replacement valve arranged at the first end of the implant for placement at the aortic valve,
a self-expandable aortic stent frame arranged at the second end of the implant for placement at the ascending aorta; and
two or more internal branches for connection to further vessels, the internal branches extending from the main body using a delivery device, the delivery device comprising;
a flush port;
a main body part for holding, inflating and/or releasing the implant;
one or more guidewires for holding, inflating and/or releasing one or more internal branches of the implant; and
an actuation mechanism for moving the implant to a delivery site,
the method comprising the steps of:
moving the implant to a delivery site by means of the delivery device;
pre-catheterizing the internal branches of the implant at the delivery site;
positioning the main body of the implant at the delivery site; and
deploying the implant at the delivery site.

31. The implant according to claim 20, wherein the replacement valve is arranged within the at least substantially cylindrical outer shape.
